(19) United States
(12) Patent Application Publication
(10) Pub. No.: US 2008/0004247 A1
(43) Pub. Date: Jan. 3, 2008

(54) Combinations of Statins with Bronchodilators

(75) Inventors: Bertil Lindmark, Lund (SE); Anders Ingemar Thoren, Lund (SE)

Correspondence Address: FISH & RICHARDSON P.C., P.O BOX 1022, MINNEAPOLIS, MN, 55440-1022, US

(73) Assignee: ASTRAZENECA AB, Södertälje (SE)

(21) Appl. No.: 11/571,869

(22) PCT Filed: Jun. 20, 2005
(86) PCT No.: PCT/GB05/02413

§ 371 Date: Jan. 9, 2007

(30) Foreign Application Priority Data
Jul. 15, 2004 (GB) 0415789.7

(51) Int. Cl. A61K 31/56; A61K 31/505; A61P 43/00

(52) U.S. Cl. 514/171; 514/275

(57) ABSTRACT

The invention provides medicaments comprising combinations of bronchodilators, glucocorticosteroids and HMG-CoA reductase inhibitors in the treatment of respiratory disorders such as chronic obstructive pulmonary disease (COPD).

### FIELD OF THE INVENTION

The invention provides medicaments comprising combinations of bronchodilators, glucocorticosteroids and HMG-CoA reductase inhibitors in the treatment of respiratory disorders such as chronic obstructive pulmonary disease (COPD).

### BACKGROUND OF THE INVENTION

Both diagnosis and management of many diseases focus, for obvious reasons, on typical criteria and manifestations, which are characteristic for that particular disease (thereby discriminating it from other entities). Examples are joint-related signs and symptoms in rheumatic arthritis (RA) and lung functions test in COPD.

However, many diseases have significant co-morbidity, which often bave been regarded as “other” diseases, since they are not unique or characteristic to the primary disease. For example, cardiovascular co-morbidity may often be viewed as unspecific and not directly linked to primary diseases such as RA, or COPD. Yet, co-morbidity may be just as important as the traditional manifestations of the primary disease, both in terms of quality of life for the patients and for the cost for society.

Chronic obstructive pulmonary disease (COPD) is a term used to describe patients with irreversible airway obstruction, usually in association with chronic bronchitis and emphysema, and epidemiologically clearly linked to smoking. COPD is characterised by both an accelerated decline in lung function and periods of acute deterioration in symptoms and exercise capacity termed exacerbations. The disease thus is serious and progressive and often leads to severe breathing disabilities, hypoxemia and eventually to death. COPD is the fourth leading cause of death in the industrialised world and exerts a heavy burden on patients, their careers, healthcare resources and society. In the western world COPD is predominantly observed in smokers, but in other parts of the world infections and in-door cooking seem to predispose. COPD is a disease where inflammation and impaired mucosal immune defence, induced by smoking, may contribute to co-morbidity. A systemic inflammation continues to be active also long after smoking cessation.

Patients with COPD are numerous and the disease is difficult to treat. Treatments exist that have effect on bronchospasm, symptoms, quality of life and exacerbations, however there is none that is able to slow down the progressive and accelerated loss of lung function. One of the primary objectives of treatment is to reduce the progression of the disease and to obtain this smoking cessation is the most important step. However, far from all COPD patients can or even wish to give up smoking and even if the patients stop smoking the airway obstruction will most often not disappear. In these cases pharmacological therapy may provide some relief. Up to date there are only a few groups of pharmacological treatments that have been tested with different results in COPD, namely bronchodilating agents and glucocortico-steroids. The bronchodilating class consists mainly of short and long-acting anticholinergics and β₂-agonists. The glucocorticosteroid treatment approach is more questioned, but with the introduction of combination therapies using the long-acting β₂-agonists such as formoterol and salmeterol together with glucocorticosteroids such as budesonide and fluticasone propionate, a new pharmacological tool has become available. In recent years combination products containing a long-acting β₂-agonist and a glucocorticosteroid e.g. formoterol/budesonide (AstraZeneca) and salmeterol/fluticasone propionate (GSK) have become available.

In addition current anti-inflammatory drugs, developed for signs and symptoms of a particular disease, may not be optimized for long-term treatment of the concomittant systemic inflammation which is hypothesized being responsible for much of the co-morbidity. Such therapy must be able to reduce an ongoing, systemic inflammation - and yet have good tolerability and safety.

### DESCRIPTION OF THE INVENTION

Many specialists express the need for new therapies for all aspects of COPD, but it is particularly important to find ways to eliminate or at least reduce the declining of the disease with time.

Several inflammatory mediators are likely to be involved in COPD as many inflammatory cells are activated. In medical practice for the treatment of e.g. asthma the influence on a single mediator has been unsuccessful in the development of new therapies. There are different mediators involved in COPD compared to asthma and therefore it is necessary to develop different drugs. Among targets for COPD have been mentioned leukotriene B₄ inhibitors, chemokine antagonists, neutrophil elastase, phosphodiesterase-4 inhibitors, cathepsins, matrix metallo-proteinases (MMPs), protease inhibitors and many others. Compelling evidence suggests that the lung damage associated with COPD results from an imbalance between proteases.

Matrix metalloproteinases are capable of degrading all of the components of the extracellular matrix of lung parenchyma including elastin, collagen, proteoglycans, laminin and fibronectin (FASEB J, 12 1075 (1998)). It has been developed some nonselective MMP lo inhibitors, but the side effects may be a problem in long-term use. More selective inhibitors of individual MMPs, such as MMP-9 and MMP-12 are now in development.

Statins are increasingly being recognized as anti-inflammatory agents. Schonbeck and Libby (Circulation, 109 (suppl. II), II-18-26 (2004)) are addressing this by reviewing in vitro and in vivo evidence regarding statins (3-hydroxy-3-methyl glutaryl coenzyme A (HMG-CoA) reductase inhibitors) as antiinflammatory agents. Any connections of use of statins in respiratory disorders of any kind are not addressed at all by these authors.

Statins are the most commonly used lipid-lowering compounds. Examples are lovastatin, rosuvastatin (Crestorm, AstraZeneca), pravastatin (Pravachol™, Bristol-Myers Squibb), simvastatin (Zocord™, Merck), itavastatin, cerivastatin, fluvastatin, atorvastatin (Lipitor™, Pfizer) and mevastatin. WO 00/48626 (Univ. of Washington) provides a composition comprising a HMG-CoA reductase inhibitor (statin) at a concentration of less than 0.1 mg and a method of treating a pulmonary disease including COPD with an aerosol formulation of statins.

EP 1 275 388 (Takeda) provides a TNF-α inhibitor (statins) for the prevention and treatment of TNF-α-associated diseases such as inflammatory diseases including asthma and COPD.

The statin cerivastatin has been shown to reduce inflammatory activity in alveolar macrophages derived from chronic bronchitis patients (Circulation 101 (2000), 1760). In a study with patients receiving statins it was shown that initiation of statin therapy was associated with a significant improvement (certain patient inclusion criteria were used) in the rate of FEV, decline that was unrelated to cigarette use factors. The prestatin baseline FEV, slope was −109.2 ml/yr and following statin therapy the slope was −46.7 ml/yr (Chest, 120 (4), suppl, p291S (2001)).

We have now found that a combination of a HMG-CoA reductase inhibitor (preferably a statin), a bronchodilator and a glucocorticosteroid given separately, sequentially or simultaneously may potentiate the effect of either component and also produce a better effect than conventional COPD treatments. The therapeutic effect may be observed with regard to the fast decline in lung function that is a hallmark of COPD, and effects may be observed regarding the systemic inflammation that is also characteristic of COPD. The long-term effect of a combination according to the invention will be conservation of lung function and putatively less co-morbidity (based on effects on the systemic inflammation).

In a first aspect the invention provides a pharmaceutical combination comprising, in admixture or separately:
(a) one or more first active ingredient which is/are a statin, a pharmaceutically acceptable salt or solvate thereof, or a solvate of such a salt,
(b) one or more second active ingredient which is/are a bronchodilator, a pharmaceutically acceptable salt or solvate thereof, or a solvate of such a salt; and optionally
(c) one or more third active ingredient which is/are a glucocorticosteroid a pharmaceutically acceptable salt or solvate thereof, or a solvate of such a salt.

The combinations of the invention can be used for the treatment of respiratory diseases such as asthma, COPD and fibrolytic diseases like systemic sclerosis, alveolitis, sarcoidosis and idiopathic pulmonary fibrosis.

The pharmacologically active agents in accordance with the present invention include statins like lovastatin, rosuvastatin (Crestorm, AstraZeneca), pravastatin (Pravachol™, Bristol-Myers Squibb), simvastatin (Zocord™, Merck), itavastatin, cerivastatin, fluvastatin, atorvastatin (Lipitor™, Pfizer) and mevastatin.

Suitable glucocorticosteroids include budesonide, fluticasone (e.g. as propionate ester), mometasone (e.g. as furoate ester), beclomethasone (e.g. as 17-propionate or 17,21-dipropionate esters), ciclesonide, loteprednol (as e.g. etabonate), etiprednol (as e.g. dicloacetate), triamcinolone (e.g. as acetonide), flunisolide, zoticasone, flumoxonide, rofleponide, butixocort (e.g. as propionate ester), prednisolone, prednisone, tipredane, steroid esters according to WO 2002/12265, WO 2002/12266 and WO 2002/88167 (I) e.g. 60α,9α-difluoro-17α-[(2-furanylcarbonyl)oxy]-11β-hydroxy-16α-methyl-3-oxo-androsta- 1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester and 6α,9α-difluoro-11β-hydroxy-16α-methyl-17α-[(4-methyl-1,3-thiazole-5-carbon 3-oxo-androsta-1,4-diene- 17β-carbothioic acid S-fluoromethyl ester, steroid esters according to DE 4129535 (II) and the like.

Preferably the bronchodilator is a long-acting β₂-agonist. Suitable long-acting β₂-agonists include salmeterol, formoterol, bambuterol, TA 2005 (chemically identified as 2(1H)-Quinolone, 8-hydroxy-5-[1-hydroxy-2-[[2-(4-methoxy-phenyl)-1-methylethyl]-amino]ethyl]-monohydrochloride, [R-(R*,R*)] also identified by Chemical Abstract Service Registry Number 137888-11-0 and disclosed in U.S. Pat. No 4.579.854 (=CBF-4226, carmoterol)), QAB149 (CAS no 312753-06-3; indacaterol), formanilide derivatives (III) e.g. 3-(4-{[6-({(2R)-2-[3-(formylamino)-4-hydroxyphenyl]-2-hydroxyethyl}amino)hexy]oxy}-butyl)-benzenesulfonamide as disclosed in WO 2002/76933, benzenesulfonamide derivatives (IV) e.g. 3-(4-{[6-({(2R)-2-hydroxy-2-[4-hydroxy-3-(hydroxy-methyl)phenyl]ethyl}amino)-hexyl]oxy}butyl)benzenesulfonamide as disclosed in WO 2002/88167, aryl aniline receptor agonists as disclosed in WO 2003/042164 and WO 2005/025555 (V), indole derivatives as disclosed in WO 2004/032921 and the like. Among the anticholinergic compounds may be mentioned ipratropium (e.g. as bromide), tiotropium (e.g. as bromide), oxitropium (e.g. as bromide), tolterodine, solifenacin (e.g. as succinate), imidafenacin, darifenacin, fesoterodine, glycopyrronium (e.g. as bromide), mepensolate (e.g. as bromide), quinuclidine derivative such 3(R)-(2-hydroxy-2,2-dithien-2-ylacetoxy)- 1-(3-phenoxypropyl)-1-azonia-bicyclo[2.2.2]octane bromide as disclosed in US 2003/0055080 and the like. Several of these compounds could be administered in the form of pharmacologically acceptable esters, salts, solvates, such as hydrates, or solvates of such esters or salts, if ally. Both racemic mixtures as well as one or more optical isomers of the above compounds are within the scope of the invention.

Suitable physiologically acceptable salts include acid addition salts derived from inorganic and organic acids, for example the chloride, bromide, sulphate, phosphate, maleate, fumarate, citrate, tartrate, benzoate, 4-methoxybenzoate, 2- or 4-hydroxybenzoate, 4-chlorobenzoate, p-toluenesulphonate, methanesulphonate, ascorbate, acetate, succinate, lactate, glutarate, tricarballylate, hydroxynaphthalene-carboxylate (xinafoate) or oleate salts or solvates thereof. The second active ingredient is preferably formoterol fumarate dihydrate or salmeterol xinafoate.

The preferred pharmacologically active statins for use in accordance with the present invention include rosuvastatin and atorvastatin. The preferred glucocorticosteroid agents include mometasone furoate, ciclesonide, zoticasone, flumoxonide, steroid (I), steroid (II), fluticasone propionate and budesonide, and even more preferred is budesonide. The preferred pharmacologically active long-acting β₂-agonist is salmeterol xinafoate, formanilide derivatives (III), benzenesulfonamide derivatives (IV) and formoterol (e.g. as fumarate dihydrate) and even more preferred is formoterol fumarate dihydrate. Among the more preferred anticholinergic agents are tiotropium, tolterodine and the quinuclidine derivatives as stated in US 2003/005580.

Preferably one active ingredient from each class is present, i.e. one statin, one bronchodilator and one glucocorticosteroid.

The preferred combinations include:atorvastatin/formoterol fumarate dihydrate
rosuvastatin/formoterol fumarate dihydrate
pravastatin/formoterol fumarate dihydrate
simvastatin/formoterol fumarate dihydrate
atorvastatin/budesonide/formoterol fumarate dihydrate, rosuvastatin/budesonide/formoterol fumarate dihydrate, rosuvastatin/ciclesonide/formoterol fumarate dihydrate,
atorvastatin/fluticasone propionate/salmeterol xinafoate, atorvastatin/ciclesonide/formoterol fumarate dihydrate,
rosuvastatin/mometasone furoate/formoterol fumarate dihydrate, and
rosuvastatin/fluticasone propionate/formoterol fumarate dehydrate.

The most preferred combinations are
rosuvastatin/formoterol fumarate dihydrate
atorvastatin/budesonide/formoterol fumarate dihydrate and
rosuvastatin/budesonide/formoterol fumarate dihydrate.

Other preferred combinations include:
Rosuvastatin/formoterol fumarate dihydrate/tiotropium bromide
Atorvastatin/formoterol fumarate dihydrate/tiotropium bromide
Atorvastatin/formoterol fumarate dihydrate/tolterodine
Rosuvastatin/tiotropium bromide
Atorvastatin/tiotropium bromide

According to the invention there is provided a combination comprising, in admixture or separately:
(a) one or more first active ingredient(s) which is/are a statin, a pharmacetucally acceptable salt or solvate thereof, or a solvate of such a salt,
(b) one or more second active ingredient(s) which is/are a bronchodilator, a pharmaceutically acceptable salt or solvate thereof, or a solvate of such a salt; and optionally
(c) one or more third active ingredient(s) which is/are a glucocorticosteroid, a pharmaceutically acceptable salt or solvate thereof, or a solvate of such a salt in the manufacture of a medicament for use in the treatment of respiratory diseases.

The invention also provides a method of treating a respiratory disease which comprises administering to the patient a therapeutically effective amount of a combination comprising, in admixture or separately:
(a) one or more first active ingredient which is/are a statin, a pharmaceutically acceptable salt or solvate thereof or a solvate of such a salt
(b) one or more second active ingredient which is/are a bronchodilator, a pharmaceutically acceptable salt or solvate thereof, or a solvate of such a salt; and optionally
(c) one or more third active ingredient which is/are a glucocorticosteroid, a pharmaceutically acceptable salt or solvate thereof, or a solvate of such a salt.

The effective dose of the components will strongly depend on the particular compound used and the mode of administration, as well as the weight and disease state of the individual being treated. An orally administered dose of the statins will generally range from about 0.01 mg to about 200 mg, preferably from 10 to 80 mg, more preferably from 5 to 40 mg; for inhalation a dose range of 0.001 mg to about 25 mg is preferred, even more preferably is a dose from 0.1 to 25 mg.

The suitable daily dose of the long-acting β₂-agonists is in the range of 1 [g to 100 mg depending on potency of each compound e.g. for formoterol the daily dose is in the range of 1 to 100 μg with the preferred dose of 3 to 48 μg (as fumarate dihydrate). The suitable daily dose for the glucocorticosteroids is in the range of 50 μg to 2000 μg, where e.g. for budesonide the daily dose is in the range of 50 μg to 1600 μg. The doses for inhalation of the anticholinergic agents are from 1 microgram to 300 micrograms, preferably for ipratropium bromide (Atroventrm, Boehringer Ingelheim) the dose is 10 to 200 microgram and for tiotropium (Spiriva™, Boehringer Ingelheim) the dose is 1 to 50 ug.

Suitably the molar ratio of the second active ingredient to the third active ingredient of from 1:2500 to 12:1.

The molar ratio of the second active ingredient to the third active ingredient is preferably from 1:555 to 2:1 and more preferably from 1:150 to 1:1. The molar ratio of the second active ingredient to the third active ingredient is more preferably from 1:133 to 1:6. The molar ratio of the second active ingredient to the third active ingredient is most preferably 1:70 to 1:4.

The components of the invention can be administered in admixture, i.e. together, or separately. When administered together the components can be administered as a single pharmaceutical composition such as a fixed combination given by e.g. inhalation. Alternatively the components can be administered separately, i.e. one after the other e.g. the statin orally and the two remaining components by inhalation. The time interval for separate administration can be anything from direct sequential (one after the other) administration to administration several hours apart.

Examples of respiratory diseases that can be treated according to the invention include asthma, chronic obstructive pulmonary disease (COPD), systemic sclerosis alveolitis, sarcoidosis, cystic fibrosis, fibrinous and pseudomembraneous rhinitis and idiopathic pulmonary fibrosis.

The invention further provides a process for the preparation of a pharmaceutical composition of the invention which comprises mixing
(a) one or more first active ingredient which is/are a statin, a pharmaceutically acceptable salt or solvate thereof, or a solvate of such a salt;
(b) one or more second active ingredient which is/are bronchodilator, a pharmaceutically acceptable salt or solvate thereof, or a solvate of such a salt; and optionally
(c) one or more third active ingredient which is/are a glucocorticosteroid, a pharmaceutically acceptable salt or solvate thereof, or a solvate of such a salt;
with a pharmaceutically acceptable adjuvant, diluent or carrier.

The therapeutically active ingredients may be administered prophylactically as a preventive treatment or during the course of a medical condition as a treatment of cure.

The pharmaceutical compositions may be administered topically (e.g. to the lung and/or airways or to the skin) in the form of solutions, suspensions, fluoroalkane aerosols and dry powder formulations; or systemically, e.g. by oral administration in the form of tablets, capsules, syrups, powders or granules, or by parenteral administration in the form of solutions or suspensions, or by subcutaneous administration or by rectal administration in the form of suppositories or foams or transdermally.

The composition used in the invention optionally additionally comprises one or more pharmaceutically acceptable additives, diluents and/or carriers. The composition is preferably in the form of a dry powder for inhalation, wherein the particles of the pharmaceutically active ingredients have a mass median diameter of less than 10 μm.

When administered separately, administration can be via alternative routes. For example the statin can be administered orally and the steroid and β-agonist can be administered in combination via inhalation, either as a powder, or aerosol formulation or as a formulation suitable for nebulisation. The compounds could be delivereed from a single chamber/cartridge but also from a two or three chambers/cartridges with separate channels.

Biological Data

As stated above COPD is a chronic disease, triggered by smoking in susceptible individuals. It is characterised by various respiratory symptoms such as breathlessness, productive cough and wheezing. These symptoms may increase sharply by acute exacerbations at various intervals. Respiratory infections are important triggers for exacerbations which can be life-threatening and have an important impact on quality of life. A number of drugs have shown some preventive effect on the incidence of exacerbations, such as inhaled corticosteroids (ICS), particularly in combination with long acting beta agonists (LABA). Synbicorto, a fixed combination of budesonide and formoterol, has been approved for treatment of COPD based on the effect of symptoms, quality of life, and prevention of severe exacerbations. This effect includes the most strict definition of severe exacerbations: Need for hospitalisation due to respiratory symptoms or need for a course of oral corticosteroids.

In post-hoc analyses of clinical long-term trials of COPD it has been observed a positive effect on FEV, decline in patients treated with statins. This effect was not seen with any other treatment including ICS. Regarding exacerbations defined as above, there was a synergistic effect of budesonide and formoterol given as Symbicort®. No effect on exacerbations by statins has been described or anticipated. To our surprise, we found that the effect of the combination of formoterol and budesonide (Symbicort) on exacerbations could be further potentiated by statins.

Methods

A meta-analysis was performed from 2 one-year clinical trials in moderate to severe COPD. Patients treated with budesonide (Pulmicort®), formoterol (Oxis®), formoterol+budesonide (Symbicort®) or Placebo were analysed with and without statins as concomitant medication. The incidence of severe exacerbations, defined as need for a treatment course of oral corticosteroids, was determined.

Results

The result of the analysis is shown in Table 1. The positive effect of the combination of formoterol and budesonide (Symbicort®) treatment vs the monocomponents (budesonide and formoterol resp.) is demonstrated and was further amplified if the patients received treatment with statins. The lowest incidence of exacerbations was seen in patients receiving Symbicort® plus statins, 0.3 per year, corresponding a 75% reduction vs the placebo group.

TABLE 1
Effect on severe COPD exacerbations by different treatments.
(Number of treated patients)

| | Incidence of exacerbations per year | |
|---|---|---|
| Treatment | EXCLUDING STATINS | PLUS STATINS |
| Placebo | 1.1 (n = 380) | 0.6 (n = 8) |
| Budesonide | 0.9 (n = 379) | 0.7 (n = 8) |
| Formoterol | 1.0 (n = 388) | 0.4 (n = 9) |
| Budesonide/formoterol | 0.7 (n = 399) | 0.2 (n = 6) |

The positive effect of a combination of formoterol and budesonide (Symbicort®) treatment on exacerbations in COPD was potentiated by statins, and the combination of formoterol and budesonide (Symbicort®) and statins gave the lowest incidence of COPD exacerbations, corresponding to a 82% reduction vs the placebo group.

The invention is illustrated by the following examples

### EXAMPLE 1

### Inhalation—Dry Powder

| Ingredients | Per dose | |
|---|---|---|
| Formoterol (as fumarate dihydrate) | 4.5 | μg |
| Budesonide | 160 | μg |
| Rosuvastatin | 1 | mg |

### EXAMPLE 2

### Inhalation—Metered Dose Inhaler

| Ingredients | Per dose | |
|---|---|---|
| Formoterol (as fumarate dihydrate) | 4.5 | μg |
| Budesonide | 160 | μg |
| Rosuvastatin | 1 | mg |
| HFA 227 | 50 | μl |

### EXAMPLE 3

### Inhalation—Dry Powder

| Ingredients | Per dose | |
|---|---|---|
| Formoterol (as fumarate dihdyrate) | 4.5 | μg |
| Budesonide | 160 | μg |
| Rosuvastatin | 1 | mg |
| Lactose | up to 1, 2, 5, 10 or 20 | mg |

### EXAMPLE 4

### Inhalation/Oral Administration

| | |
|---|---|
| Ingredients | Per dose/tablet |

Aerosol formulation

| | | |
|---|---|---|
| Formoterol (as fumarate dihydrate) | 4.5 | μg |
| Budesonide | 160 | μg |

A tablet formulation

| | | |
|---|---|---|
| Rosuvastatin | 10 | mg |

### EXAMPLE 5

### Inhalation/Oral Administration

| | |
|---|---|
| Ingredients | Per dose/tablet |

Aerosol formulation

| | | |
|---|---|---|
| Formoterol (as fumarate dihydrate) | 4.5 | ug |
| Budesonide | 160 | ug |

A tablet formulation

| | | |
|---|---|---|
| Rosuvastatin | 20 | mg |

1. A pharmaceutical combination comprising; in admixture or separately:
(a) one or more first active ingredients which is/are a statin, a pharmaceutically acceptable salt or solvate thereof, or a solvate of such a salt.
(b) one or more first active ingredient which is/are a bronchodilator, a pharmaceutically acceptable salt or solvate thereof, or a solvate of such a salt, and optionally
(c) one or more third active ingredient which is/are a gluococorticosteroid, a pharmaceutically acceptable salt or solvate thereof, or a solvate of such a salt.

2. A combination according to claim 1 wherein the statin(s) is/are selected from lovastatin, rosavastatin, pravastatin, simvastatin, itavastatin, cerivastatin, fluvastatin, atorvastin and roevastatin.

3. A combination according to claim 1 wherein the statin is rosuvastatin.

4. A combination according to claim 1 wherein the statin is atovastatin.

5. A combination according to claim 1 wherein the bronchodilator(s) is/are a long-acting β₂-agonist.

6. A combination according to claim 1 wherein the long-acting β_{2+L-AGONIST(S) is/are selected frp, salmeterol, formoterol, bambiterol,} 2(IH)-Quinolone, 8- hydroxy-5-[1-hydroxy-2[[2-(4-methoxy-phenyl)-1-methylethyl]-amino]ethyl]-monohydrochloride, [R-(R*,R*)], 3-(4-{[δ-{3-(formylamino)-4-hydroxyphenyl]-2-hydoxyethyl)amino)hexyl]oxy}-butyl)-benzenesulfonamide or 3-(4{[6 -([(2R)-2-hydroxy-2-[4-hydroxy-3-(hydroxy-methyl)phenyl]ethyl}amino) -hexyl]oxybutylibenzensifonamide and pharmaceutically acceptable salts or solvates thereof, or a solvates of salts.

7. A combination according to claim 1 wherein the long-acting β₂-agonist is formoterol or a pharmaceutically acceptable salt of solvate thereof, or a solvate of such a salt.

8. A combination according to claim 1 wherein the long acting +62₂-agonist is formoterol fumarate dehydrate.

9. A combination according to any of claim 1 wherein the bronchodilator(s) is/are an antocholinergic agents or a pharmaceutically acceptable salt or solvate thereof, or a solvate of such a salt.

10. A combination according to claim 1 in which the anticholinergic agent(s) is/are selected from ipratropium (e.g. as bromide), tiotropium (e.g. as bromide), oxitrpium (e.g. as bromide), tolterodine, soliferacin (e.g. as succinate), imdafenacin, darlfenacia, fesotemdine, glycoprronium (e.g as bromide), mepesolate (e.g. as bromide), quinaclidine derivative such 3(R)-(2-hydroxy-2.2-dithien-2-ylacetoxyl)-1-(3 - phenoxypropyl)-1-azenia-bicyclo[2.2.2]octane bromide and pharmaceutically acceptable salts thereof, or a solvates of salts.

11. A combination according to claim 10 in which the anticholinergic agent is tiotropium bromide.

12. A combination according to claim 1 in which the glucocorticosteroid(s) is/are selected from budesonide, fluticasone, mometasonem beclomethasone, ciclesonide, loteprednol, etiprednol, triamcinoline, flunisolide, zoticasone, flumoxonide, refleponide, butixocort, predniosoline, prednisone, tipredane, 6α,9α-difluoro-17α-[(2-furanylcarbonyl)oxy]-11β-hydroxy-16α-methyl-3-oxo-17α-propionyloxy -androsta-1,4-diene-17β-carbothioic acid S-fluoromethyl ester, and pharmaccutically acceptable salts thereof.

13. A combination according to claim 1 in which the glucocorticosteroid is budesonide.

14. A combination according to claim 1 for use in the treatment of respiratory diseases.

15. A combination according to claim 1 for use in the treatment of COPD.

16. A method of treating a respiratory disease which comprises administering to the patient a therapeutically effective amount of a combination comprising, in admixture or separately:
(a) one or more first active ingredient which is/are a statin, a pharmaceutically acceptable salt or solvate thereof or a solvate of such a salt.
(b) one or more second active ingredient which is/are a bronchodilator, a pharmaceutically acceptable salt or solvate thereof, or a solvate of such a salt;
(c) one or more third active ingredient which is/are a glucocorticosteroid.

17. A method according to claim 16 wherein the disease is COPD.
